(19) United States
(12) Patent Application Publication
(10) Pub. No.: US 2009/0204185 A1
(43) Pub. Date: Aug. 13, 2009

(54) PHOTOTHEREAPY DEVICE

(75) Inventors: Margreet De Kok, Eindhoven (NL); Liesbeth Van Pieterson, Heeze (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/304,281

(22) PCT Filed: Jun. 4, 2007
(86) PCT No.: PCT/IB2007/052086

§ 371 Date: Dec. 11, 2008

(30) Foreign Application Priority Data
Jun. 14, 2006 (EP) 06115458.9

(51) Int. Cl. A61N 5/08

(52) U.S. Cl. 607/88

(57) ABSTRACT

The present invention relates to a phototherapy device (1), a medical system (7), and a phototherapy method for prophylaxis of infections at an entrance site (2) of a catheter (3) or the like during intravascular access, e.g. infusion or the like. In particular, a phototherapy device (1) is suggested, said device (1) comprising at least one light emitter (8, 14, 17) for applying red and/or infrared and/or UV light to a patient at the entrance site (2), said light emitter being connected to or part of an attachment medium (6, 16), said attachment medium (6, 16) being adapted to attach medical equipment (3) which is used for and/or during the intravascular access, e.g. infusion equipment, to the skin (4) of the patient.

The present invention relates to a phototherapy device, a medical system, and a phototherapy method for prophylaxis of infections at an entrance site of a catheter or the like during intravascular access, e.g. infusion or the like.

Intravascular catheters are indispensable in modern-day medical practice, particularly in intensive care units (ICUs). Although such catheters provide necessary vascular access, their use puts clients at risk of local and systemic infectious complications, including local site infection, catheter-related bloodstream infections (CRBSI), septic thrombophlebitis, endocarditis, and other infections.

The incidence of CRBSI varies considerably from type of catheter, frequency of catheter manipulation, and client-related factors. Peripheral venous catheters are the devices most frequently used for vascular access. Although the incidence of local or bloodstream infections (BSIs) associated with peripheral venous catheters is usually low, serious infectious complications produce considerable annual morbidity because of the frequency with which such catheters are used. However, the majority of serious catheter-related infections are associated with central venous catheters (CVCs), especially those that are placed in clients in ICUs.

Some catheters are inserted in urgent situations, during which optimal attention to aseptic technique may not be feasible. Certain catheters (e.g. pulmonary artery catheters, peripheral arterial catheters) can be accessed many times a day for haemodynamic measurements or to obtain samples for laboratory analysis, augmenting the potential for contamination and subsequent clinical infection.

The intravenous (IV) cannula offers direct access to a patient's vascular system and provides a potential route for entry of microorganisms into that system. These organisms can cause serious infection if they are allowed to enter and proliferate in the IV cannula, insertion site, or IV fluid.

IV therapy-related bacteremia is a potential cause of serious illness or death for patients. Additional cannula-related complications which can occur with or without fever or bacteremia include phlebitis, occult IV-site infection, cellulites, and purulent thrombophlebitis.

Treatment of infections caused by intravenous catheters is, depending on the origin of the infection, either microbiological by bacteria or sterile. Standard treatment of the “sterile infection” is application of alcohol and dedicated cream to stimulate blood perfusion of the tissue surrounding the affected arteries and veins. In the case of a microbiological origin of the infection, antibiotics supplied either systemic or in cream are needed. The bacteria causing the infection have to be identified in order to know what antibiotics should be used. Problematic at this point is the fact that determination of the bacterial origin takes several days due to cell proliferation. In some situations this delay is detrimental; in anticipation of this analysis broad-spectrum antibiotics are administered to the patient. The broad-spectrum antibiotics are a burden to bodies of critically ill patients and if the appropriate antibiotics are not included, the infection could lead to the patient's death.

Any method to prevent the incidence of infection due to catheters and cannula would mean an improvement in critical and post-operational care.

The standard measures taken to prevent infections in the case of use of intravenous catheters and cannula are antiseptics used at the introduction, replacement and any handling of the catheters. All materials used including the fluids to be administered should be sterile and the clinical staff should carefully monitor the site of introduction. The site of introduction is protected e.g. by a thin foil, that keeps out water, dirt and germs, to minimize invasion of pathogens via the skin.

Nevertheless infections occur regularly. When infection occurs, the patient is treated with antibiotics and/or cream. However, it would be better to prevent infection in the first place.

It is an object of the present invention to provide a technique for preventing infection of an entrance site of a catheter or the like during intravascular access, e.g. infusion or the like.

This object is achieved according to the invention by a phototherapy device comprising at least one light emitter for applying red and/or infrared and/or ultraviolet (UV) light to a patient at the entrance site, said light emitter being connected to or part of an attachment medium, said attachment medium being adapted to attach medical equipment to the skin of a patient. In particular, said attachment medium is adapted to attach medical equipment to the skin of the patient which is used for and/or during the intravascular access, e.g. infusion equipment. The term light emitter is to be understood as outcoupling medium, i.e. a medium adapted to couple out light to a target. The light emitter may comprise an internal light source. However, in other cases the light emitter may not comprise an internal light source, i.e. the light emitter is connected to an external light source.

The object of the present invention is also achieved by a medical system comprising medical equipment, and a phototherapy device as described above, the attachment medium of said phototherapy device attaching the medical equipment to a patient's skin. In particular, said medical equipment is used for and/or during the intravascular access, e.g. infusion equipment.

The object of the present invention is also achieved by a phototherapy method comprising the steps of attaching a phototherapy device as described above to the skin of a patient, and applying, via said phototherapy device, red and/or infrared light to the patient at the entrance site.

The object of the present invention is also achieved by the use of a phototherapy device as described above for prophylaxis of infections at an entrance site of a catheter or the like during intravascular access.

If the body is entered for longer periods, for example days, via the vascular system in order to introduce medicine, food and to allow blood analysis, infection is an often-encountered complication. A core idea of the invention is to prevent, a priori, an infection at an entrance site of a catheter or the like during intravascular access by stimulating the blood (micro) circulation at the entrance site. For this purpose, according to the present invention, the technique of phototherapy with (red/infrared) light is used. Red and near infrared light, i.e. light with a wavelength between 600 and 800 nm (red light) and between 0.75 and 1.4 μm (near infrared light), stimulates perfusion of the tissue by light-induced blood vessel and lymph vessel vasodilatation. The same applies for UV light with a wavelength between 200 and 380 nm. Stimulated perfusion will reduce inflammation and transport white blood cells to the location where the risk of infection is highest. Further, the immune system is stimulated to protect the body from inflammation by induced NO synthesis by light. In other words, by stimulation of perfusion of the penetrated tissue the immune system is activated and the number of complications is anticipated to be reduced. As for patients in critical care these complications are often life threatening, any reduction of the incidence of infection is valuable. For less critical patients recovery after surgery can be accelerated if no complications arise from the intravenous therapy.

The entrance site includes the opening in the skin (puncture) through which the catheter or the like passes. The entrance site may also include the region, i.e. skin and tissue, surrounding the opening. The present invention can be used for each type of infusion, in particular for infusions in intensive care, perioperational treatment or hemodialysis, e.g. using central venous catheters, pulmonary artery catheters, or peripheral arterial catheters or the like. A catheter may include any conduit through which fluids or mechanical devices pass into or out of the body. For example, a standard injection needle, a blood sample needle, a cannula, a trocar sheath, an introducer, or a shunt may be considered a catheter. The present invention can also by applied to catheters which do not pass through an opening in the skin but though a natural opening of the patient's body. It should be appreciated that, according to the present invention, a region of tissue or a catheter entrance site that is irradiated may be that of either a person or an animal.

These and other aspects of the invention will be further elaborated on the basis of the following embodiments which are defined in the dependent claims.

An attachment medium according to the invention may be disposed of after it has been used for a sufficient time and, if necessary, another may be readily applied. In this way the invention provides a user-friendly and hygienic use of phototherapy. The attachment medium can be dedicated to the type of skin surface. The attachment medium may be flexible in shape and size enabling it to be applied to practically any outer part of the body. According to a preferred embodiment of the invention, the attachment medium is a bandage or a foil or plaster or the like. Preferably, elastomeric material is used for the attachment medium, such as neoprene, natural rubber, silicone rubber, or a thermoplastic elastomer. Such media are already used in medical environments, and fulfill all medical demands regarding safety etc. Preferably, a self-adhering attachment medium is employed for ease of use, e.g. an adhesive tape or foil. Preferably, a self-adhering foil is used to adhere the medical equipment to the skin. Such a foil is preferably made of a light-guiding flexible material, e.g. a polydimethyl siloxane (PDMS) polymer. Additionally, such a foil can be used at the same time to cover the entrance site in order to prevent invasion of pathogens via the skin. The use of a foil allows easy implementation of the present invention.

According to another preferred embodiment of the invention, the light emitter comprises a red and/or infrared and/or UV light source. In other words, the light source is directly connected to or part of the attachment medium. This leads to a well-manageable, integrated phototherapy device. The light path from the light source to the light emitter is very short. Thus, there is only little attenuation during the light transport and extensive cabling or wiring is not necessary. In a preferred embodiment, the light emitter is identical to the light source, i.e. there is only a light source, which serves at the same time as light emitter. This is the case, for example, if an OLED (Organic Light Emitting Diode) is used as an intrinsic large-area light source, which will not need a light guide or diffuser or the like.

Alternatively, according to another preferred embodiment of the invention, the light emitter is connectable to a red and/or infrared and/or UV light source. In other words, the light source is provided outside the attachment medium and coupled to the light emitter. This leads to a very light and small phototherapy device. Furthermore, the attachment medium can be more flexible, thus allowing to attach even irregularly formed medical equipment. Preferably, a LED (Light Emitting Diode) is used as a light source and a light guide and/or a diffuser is employed as a light emitter.

If appropriate, the attachment medium is adapted in such a way that it serves as a waveguide to couple the light from an internal or external light source into the light emitter. In other words, additional light-guiding cables to connect the light source and the light emitter are not necessary.

As already described above, according to another preferred embodiment of the invention, a LED light source is used. Light source and attachment medium can be integrated in form of a “photonic textile”, i.e. fabrics that contain a lighting system, such as an LED system. In case the light source is integrated into the attachment medium, a flexible and/or transparent OLED (Organic LED) light source is preferably employed. A benefit of a transparent OLED is easy visual inspection of the entrance site. Instead of a transparent OLED, a reflective OLED is preferably used to direct a large amount of light to a patient's skin. If an OLED is used, preferably an OLED foil is used as a single integrated device, serving as light source, light emitter and attachment medium at the same time. Detailed information about the technical features of an OLED device is described in European patent application No. 05110643.3, which is incorporated herein by reference. However, other light sources can be used, e.g. commonly known infrared or UV lamps.

The phototherapy device as described can be used in different ways. According to a preferred embodiment of the invention, the light emitter is adapted to be positioned on or directly above the patient's skin. In this case, the skin of the patient, in particular the tissue surrounding the entrance site of the catheter, the cannula or the like, will be treated with light.

According to another preferred embodiment of the invention, the light emitter is adapted to be positioned on a receiving part of medical equipment outside the patient's body. Said medical equipment comprises an emitting part, which is connected to the receiving part, and which is adapted to enter the patient's body. In other words, the light will be transmitted from the light emitter of the phototherapy device to the receiving part of medical equipment, and from said receiving part to another part of the medical equipment, which is located inside the patient's body, i.e. beneath the skin, near the entrance site. In this case, the medical equipment itself or parts of the medical equipment are made of an appropriate material and serve as a light guide for guiding light into the patient's body, in particular inside the patient's arteries. The internal use of light is preferably applied in case of phlebitis.

Both the external and the internal treatment can be accomplished with a phototherapy device independent of whether an internal or external light source is used. The light source is controlled by means of a control unit, which again can be provided internally or externally. The control unit controls the driving conditions of the light source, e.g. wavelength, brightness, color, pulse duration, intensity, etc. Preferably, all control parameters can be changed by means of adjustment means, e.g. by means of adjusting knobs outside the control unit or by means of control commands to be delivered to the control unit via a communication path, e.g. via a wireless communication network. The adjustment of control parameters via control commands is preferred, since by this means it can be assured, e.g. using passwords etc., that only authorized persons can change the control parameters.

According to another preferred embodiment of the invention, at least one sensor is provided, which is adapted to obtain a number of patient conditions. Examples of patient data to be obtained are temperature and skin color. Furthermore, chemical sensing can be applied, e.g. an NO sensor can be integrated into the attachment medium. Such additional monitoring can be used next to the visual and manual inspection by the clinical staff.

Furthermore, a control unit is provided, which is connected to said sensor. The control unit is adapted to control the light emission of the light source and/or the emitted wavelength depending on the obtained patient condition data. Preferably two or more different (internal and/or external) light sources are provided and the control unit is adapted to switch between light sources depending on the patient condition data. Control unit, sensor(s) and light source(s) are connected to each other. Both the number of sensors and the control unit can be implemented as internal or external units, i.e. both the number of sensors and the control unit can be integrated into or attached to the attachment medium or provided in a remote place, e.g. in a pocket of the patient's clothing. A power source is provided either externally or internally to power the control unit and the light source(s). Preferably, batteries are used to provide a DC power source. In this case it is preferable, but not necessary, to locate the batteries in a remote location connected by a power cable in order to minimize the size and weight of the phototherapy device.

Preferably, the control unit is adapted to switch between a first wavelength, e.g. IR light, and a second wavelength, e.g. UV light, depending on the sensor data. This is advantageous in case of bacterial infection. The light therapy can then be combined with a disinfecting light therapy with light of a shorter wavelength (e.g. UV with a wavelength between 200 and 380 nm) to kill or inactivate microorganism. Preferably, a short pulse or flash of UV light is given at the time of introduction of the injection needle, and later on if required. Such a combined phototherapy device can be used both for preventing infections and for treating infections, if they occur.

These and other aspects of the invention will be described in detail hereinafter, by way of example, with reference to the following embodiments and the accompanying drawings; in which:

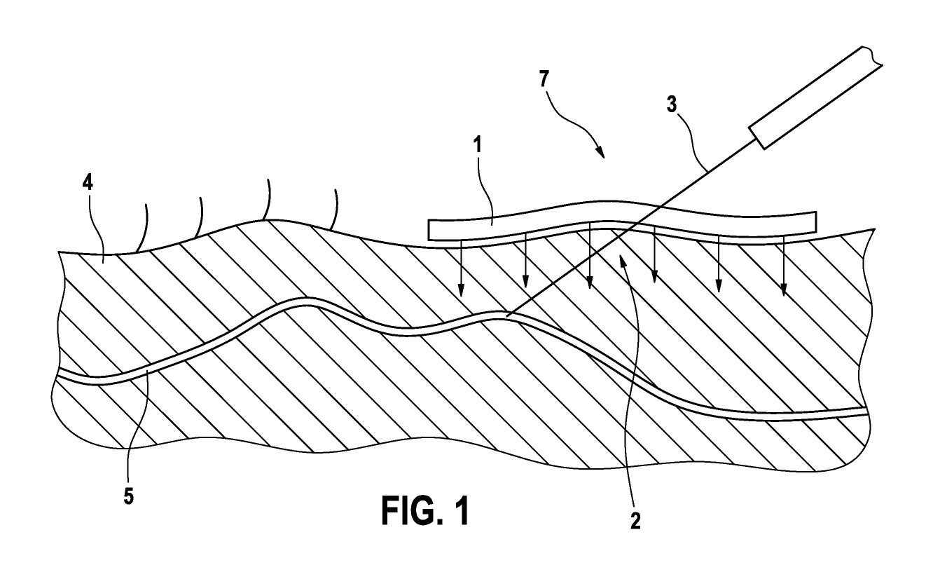

FIG. 1 shows a schematic illustration of a phototherapy device used during intravascular access,

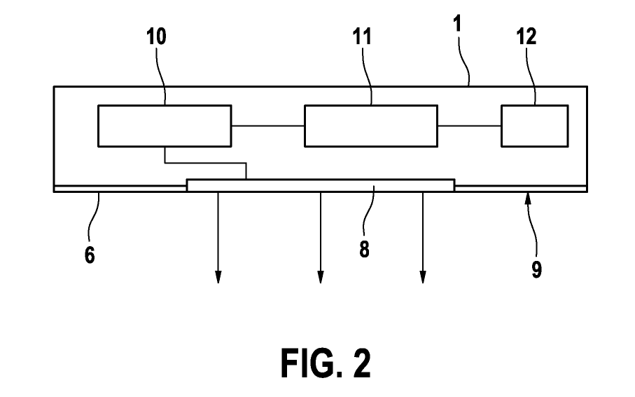

FIG. 2 shows a schematic block diagram of a phototherapy device according to the invention,

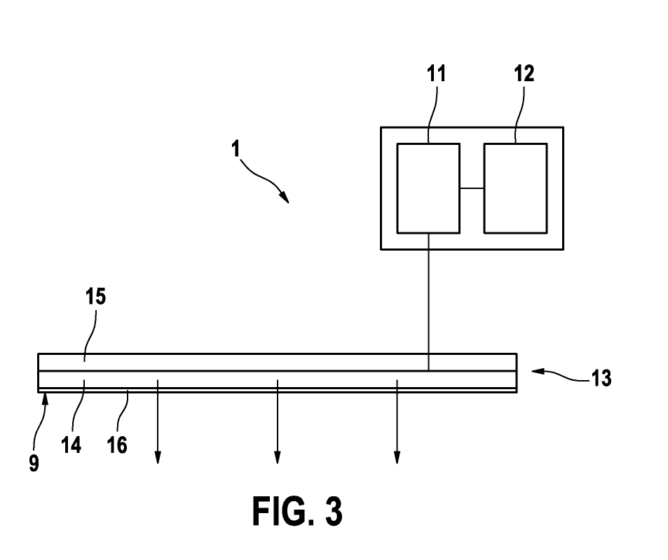

FIG. 3 shows a schematic block diagram of a phototherapy device according to the invention,

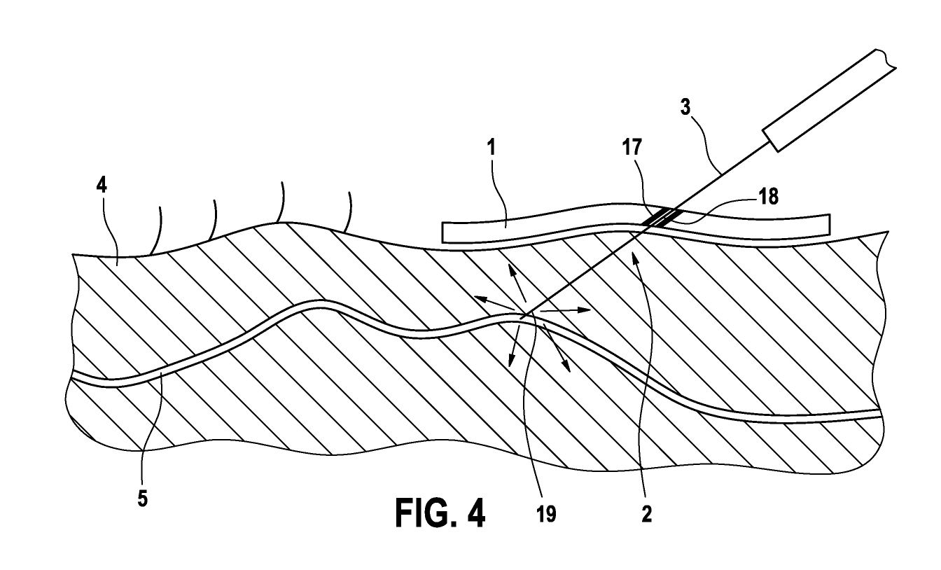

FIG. 4 shows a schematic illustration of a phototherapy device used during intravascular access, and

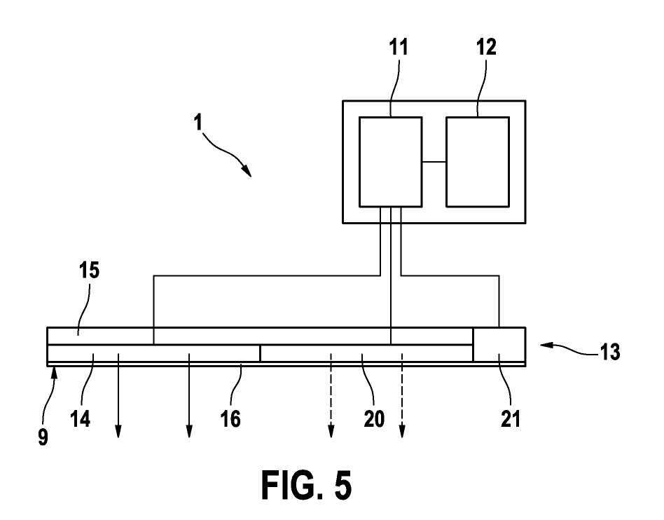

FIG. 5 shows a schematic block diagram of a phototherapy device according to the invention.

FIG. 1 illustrates a phototherapy device 1 according to the present invention, which is used for prophylaxis of infections at an entrance site 2 of a cannula 3 during intravascular access. The cannula 3 penetrates the patient's skin 4 and punctures an artery 5 of the patient. The phototherapy device 1 comprises an attachment medium 6, which contacts the cannula 3 and attaches the cannula 3 to the patient's skin 4. Phototherapy device 1 and cannula 3 form a medical system 7 according to the invention. The attachment medium 6 comprises a light emitter 8 for applying near infrared light (see arrows) to a patient's skin 4 at the entrance site 2. The light emitter 8 may be a flat display member or the like. Instead of a cannula 3, a catheter or another device may be used to illustrate the invention.

An embodiment of the invention is illustrated in FIG. 2. The light emitter 8, which comprises a diffuser, is located at the lower surface 9 of the phototherapy device 1 for emitting near infrared light (see arrows). The light emitter 8 is connected to an infrared light source 10, e.g. an infrared lamp. The light source 10 is connected to a control unit 11. The control unit 11 is connected to a power supply 12. All components are embedded in the phototherapy device 1. The phototherapy device 1 is designed in the form of a flexible plaster. It comprises an attachment medium 6, namely the adhesive part of the plaster. The attachment medium 6 forms the lower surface 9 of the phototherapy device 1 and surrounds the light emitter 8.

Another embodiment of the invention is illustrated in FIG. 3. The phototherapy device 1 comprises a flexible foil 13. The foil 13 comprises an OLED 14 on a flexible subcarrier 15 or substrate. For example, a textile or plastic subcarrier can be used. The OLED 14 forms the lower part of the foil 13, and the subcarrier 15 forms the upper part of the foil 13. The OLED 14 works as an integrated light source and light emitter for near infrared light (see arrows). The OLED 14 is self-adhering, i.e. the lower surface of the OLED 14 is coated with a transparent adhesive 16, serving as an attachment medium for attaching the cannula 3 onto the patient's skin 4. The foil 13 is connected to an external housing, which comprises the control unit 11 and the power supply 12.

In both embodiments of the invention described above, the light emitter 8, 14 is adapted to be positioned on the patient's skin 4. In another embodiment of the invention, as illustrated in FIG. 4, the light emitter 17 in form of a light guide is adapted to be positioned on a receiving part 18 of the cannula 3 outside the patient's body. The receiving part 18 is connected to another part 19 of the cannula 3, which enters the patient's body. The infrared light is guided from the light emitter 17 to the receiving part 18, and from the receiving part 18 of the cannula 3 to the emitting part 19 inside the patient's body. Inside the patient's body the near infrared light (see arrows) is emitted to the tissue underneath the entrance site 2.

Another embodiment of the invention is illustrated in FIG. 5. The phototherapy device 1 is built similarly to the device shown in FIG. 3. However, there are two different combined light emitter/light source components 14, 20 embedded in the foil 13. The first component 14 is adapted to emit infrared light, the second component 20 is adapted to emit UV light. Both components 14, 20 are connected to a control unit 11. Additionally, a sensor unit 21 is provided as part of the foil 13. The sensor unit 21 is adapted to obtain patient condition data, e.g. the skin temperature at the entrance site. Temperature data are transmitted to the control unit 11, which switches the combined light emitter/light source components 10, 20 between near infrared light (arrows) and UV light (dotted line arrows). In other words, the wavelength of the light used for phototherapy is changed depending on the sensed temperature. Alternatively, or in addition to the automatic wavelength selection, the wavelength can be controlled externally by a physician or other skilled person.

The control unit 11 may comprise functional modules or units in order to carry out the controlling tasks. The functional modules or units are implemented in form of hardware, software or a combination of both. In particular, the control unit 11 comprises a microprocessor, which is adapted for performing all tasks of computing the obtained patient data as well as assessing results and determining control instructions to control the light sources. This is achieved according to the invention by means of computer software comprising computer instructions adapted for carrying out these tasks, when the software is executed in the microprocessor.

As described above, the present invention can be implemented using different light systems. Preferred examples of such light systems are OLEDs used as an intrinsic large-area light source, LEDs with a light guide if used outside the active area, and LEDs with a diffuser if used inside the active area. A LED with light guide may be implemented as a LED light source connected to a light-guiding medium like fiber of PDMS sheet. In this case, the light is guided to the spot of therapy by means of the light guide. A LED with diffuser may be implemented as a number of inorganic LEDs, which serve as point sources, the light of which is spread over the active area to be treated by means of a diffuser in order to achieve a continuous light intensity. The diffuser is preferably used as a spacer to ensure that the right humidity at the outer skin does not create an environment where bacteria would be promoted to grow.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfill the functions of several means recited in the claims. Any reference signs in the claims shall not be construed as limiting the claim concerned.

### REFERENCE NUMERALS

1 phototherapy device
2 entrance site
3 cannula
4 skin
5 artery
6 attachment medium
7 medical system
8 light emitter
9 lower surface
10 IT light source
11 control unit
12 power supply
13 foil
14 OLED
15 subcarrier
16 adhesive
17 light emitter
18 receiving part
19 emitting part
20 UV light source
21 sensor unit

1. A phototherapy device (1) for prophylaxis of infections at an entrance site (2) of a catheter (3) or the like during intravascular access,
comprising at least one light emitter (8, 14, 17) for applying red and/or infrared and/or UV light to a patient at the entrance site (2),
said light emitter (8, 14, 17) being connected to or part of an attachment medium (6, 16), said attachment medium (6) being adapted to attach medical equipment (3) to the skin (4) of the patient.

2. The phototherapy device (1) as claimed in claim 1, characterized in that the attachment medium (6, 16) is a bandage, a foil or a plaster.

3. The phototherapy device (1) as claimed in claim 1, characterized in that the light emitter (8, 14, 17) comprises a red and/or infrared and/or UV light source (14).

4. The phototherapy device (1) as claimed in claim 1, characterized in that the light emitter (8, 14, 17) is connectable to a red and/or infrared and/or UV light source (10).

5. The phototherapy device (1) as claimed in claim 1, characterized by a LED light source, preferably an OLED light source.

6. The phototherapy device (1) as claimed in claim 1, characterized in that the light emitter (8, 14) is adapted to be positioned on the patient's skin (4).

7. The phototherapy device (1) as claimed in claim 1, characterized in that the light emitter (17) is adapted to be positioned on a receiving part (18) of medical equipment (3) outside the patient's body, another part (19) of which is adapted to enter the patient's body.

8. A medical system (7), comprising
medical equipment (3), and
a phototherapy device (1) as claimed in claim 1, the attachment medium (6, 16) of said phototherapy device (1) attaching the medical equipment (3) to a patient's skin (4).

9. A phototherapy method for prophylaxis of infections at an entrance site (2) of a catheter (3) or the like during intravascular access, said method comprising the steps of:
attaching a phototherapy device (1) as claimed in claim 1 to the skin (4) of a patient, and
applying, via said phototherapy device (1), red and/or infrared and/or UV light to the patient at the entrance site (2).

10. The phototherapy method as claimed in claim 9, characterized by the further steps of:
obtaining patient condition data by means of a sensor unit (21) or the like, and
controlling the light source (14, 20) and/or selecting the wavelength of the light to be applied to the patient, in dependence on the obtained patient condition data, by means of a control unit (11).

11. Use of a phototherapy device (1) as claimed in claim 1 for prophylaxis of infections at an entrance site (2) of a catheter (3) or the like during intravascular access.
